(19) United States
(12) Patent Application Publication
(10) Pub. No.: US 2026/0078384 A1
(43) Pub. Date: Mar. 19, 2026

(54) SYSTEMS, METHODS, AND COMPOSITIONS FOR AN INDUCIBLE PROMOTER SYSTEM

(71) Applicant: Science Corporation, Alameda, CA (US)

(72) Inventors: Alan Mardinly, Alameda, CA (US); Satoru Miura, Alameda, CA (US); Amanda Tose, Alameda, CA (US)

(73) Assignee: Science Corporation, Alameda, CA (US)

(21) Appl. No.: 19/258,604

(22) Filed: Jul. 2, 2025

Related U.S. Application Data
(60) Provisional application No. 63/666,918, filed on Jul. 2, 2024.

(51) Int. Cl. C12N 15/63; C12N 15/64

(52) U.S. Cl. CPC C12N 15/635; C12N 15/64; C12N 2830/002; C12N 2830/15

(57) ABSTRACT

In variants, an inducible promoter system, can include: a gene of interest; an operator site; a first nucleotide sequence encoding a reverse cumate transcriptional activator; and a second nucleotide sequence encoding a cumate repressor. In a specific example, the cumate repressor can bind to the operator site to repress transcription of the gene of interest and the reverse transcriptional activator can bind to the operator site in the presence of an activator to activate transcription of the gene of interest.

### CROSS-REFERENCE TO RELATED APPLICATIONS

This application claims the benefit of U.S. Provisional Application No. 63/666,918 filed 2 Jul. 2025, which is incorporated in its entirety by this reference.

### INCORPORATION BY REFERENCE OF A SEQUENCE LISTING

A Sequence Listing is provided herewith as a Sequence Listing.xml file. The Sequence Listing, created on 2 Jul. 2025, is identified as “SCIX-P11-US-SEQ-LST.xml” and is 85,829 bytes in size. The content of the Sequence Listing is incorporated in its entirety herein by this reference.

### TECHNICAL FIELD

This invention relates generally to the genetic regulation field, and more specifically to new and useful systems, methods, and compositions for an inducible promoter system in the genetic regulation field.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic representation of a variant of the inducible promoter system.

FIG. 2 is a schematic representation of an example of an activator sequence.

FIG. 3 is a schematic representation of an example of a repressor sequence.

FIG. 4A depicts a first example of an inducible promoter system, including a reverse cumate transcriptional activator (rcTA) and a (wild-type) cumate repressor (CymR), with the same CymR domains (e.g., DNA binding domain, cumate binding domain, dimerization domain, etc.) across rcTA and CymR.

FIG. 4B depicts a second example of an inducible promoter system, including a reverse cumate transcriptional activator (rcTA) and a cumate transcriptional silencer (cTS) (e.g., a fusion protein containing a cumate repressor and a silencer domain), with the same CymR domains across rcTA and cTS.

FIG. 4C depicts a third example of an inducible promoter system, including a reverse cumate transcriptional activator (rcTA) and a cumate transcriptional silencer (cTSx), with different CymR domains across rcTA and cTSx.

FIG. 4D depicts a fourth example of an inducible promoter system, including a reverse cumate transcriptional activator (rcTA) and a cumate transcriptional silencer hybrid (cTSh), with the same CymR DNA binding domain across rcTA and cTSh and with different CymR cumate binding domains and/or dimerization domains across rcTA and cTSh.

FIG. 5A-5B depict examples of vector designs for the inducible promoter system depicted in FIG. 4A.

FIG. 5C depicts an example of a vector design for the inducible promoter system depicted in FIG. 4B.

FIG. 5D depicts an example of a vector design for the inducible promoter system depicted in FIG. 4C.

FIG. 5E depicts an example of a vector design for the inducible promoter system depicted in FIG. 4D.

FIG. 6 depicts an example sequence for a reverse cumate transcriptional activator (rcTA) (e.g., with a portion of the sequence matching all or a portion of the CymR sequence of the Pseudomonas putida strain F1).

FIG. 7A depicts an example sequence for a cumate repressor CymR (e.g., substantially matching the CymR sequence of the Pseudomonas putida strain F1).

FIG. 7B depicts an example sequence for a cumate transcriptional silencer (cTS) (e.g., a fusion protein containing a cumate repressor) with a portion substantially matching the CymR sequence of the Pseudomonas putida strain F1.

FIG. 7C depicts an example sequence for a cumate transcriptional silencer (cTSx) with a portion substantially matching the CymR sequence of the Paraburkholderia xenovorans strain LB400.

FIG. 7D depicts an example sequence for a cumate transcriptional silencer hybrid (cTSh), including: a DNA binding domain sequence substantially matching the CymR sequence of the Pseudomonas putida strain F1, and a cumate binding domain sequence and/or dimerization domain sequence substantially matching the CymR sequence of the Paraburkholderia xenovorans strain LB400.

FIG. 7E depicts an example sequence for a cumate transcriptional silencer hybrid (cTSh), including: a DNA binding domain sequence substantially matching the CymR sequence of the Paraburkholderia xenovorans strain LB400, and a cumate binding domain sequence and/or dimerization domain sequence substantially matching the CymR sequence of the Pseudomonas putida strain F1.

FIGS. 8A and 8B depict examples of sequences for a regulatory element, including a promoter and an operator site (e.g., an operator site configured to interface with the DNA binding domain of CymR from the Pseudomonas putida strain F1).

FIGS. 9A and 9B depict examples of sequences for a regulatory element, including a promoter and an operator site (e.g., an operator site configured to interface with the DNA binding domain of CymR from the Paraburkholderia xenovorans strain LB400).

FIG. 10 depicts a specific example of the method.

FIG. 11A shows a doxycycline dose-dependent increase in induced green fluorescent protein (GFP) expression for a set of cells transfected with a TRE-system controlling expression of GFP.

FIG. 11B shows a cumate dose-dependent increase in induced GFP expression for a set of cells transfected with a cumate-inducible system (e.g., including an rcTA with a portion of the sequence matching all or a portion of the CymR sequence of the Pseudomonas putida strain F1, and cTSx with a portion substantially matching the CymR sequence of the Paraburkholderia xenovorans strain LB400) controlling expression of GFP.

FIG. 12A-12F shows images of the GFP (green) for the experiment shown in FIG. 11B.

FIG. 13A-13E images of the GFP (green) for the experiment shown in FIG. 11A.

FIG. 14A shows expression of GFP and BFP for a control set of cells, without a cumate-inducible system or a TRE system.

FIG. 14B shows expression of GFP and BFP for a set of cells transfected with a cumate-inducible system (e.g., including an rcTA with a portion of the sequence matching all or a portion of the CymR sequence of the Pseudomonas putida strain F1, and cTSx with a portion substantially matching the CymR sequence of the Paraburkholderia xenovorans strain LB400) controlling expression of GFP and transfected with a TRE-system controlling expression of BFP, without the addition of cumate or doxycycline.

FIG. 14C shows images of GFP and BFP for the set of cells in FIG. 14B with the addition of cumate (100 μg/mL) and doxycycline (0.025 μg/mL).

FIG. 14D shows images of GFP and BFP for the set of cells in FIG. 14B with the addition of cumate (100 μg/mL) and without doxycycline.

FIG. 14E shows images of GFP and BFP for the set of cells in FIG. 14B with the addition of doxycycline (0.025 μg/mL) and without cumate

FIG. 15A shows images of GFP (green) and BFP (blue) in the experiment shown in FIG. 14B.

FIG. 15B shows images of GFP (green) and BFP (blue) in the experiment shown in FIG. 14D.

FIG. 15C shows images of GFP (green) and BFP (blue) in the experiment shown in FIG. 14E.

FIG. 15D shows images of GFP (green) and BFP (blue) in the experiment shown in FIG. 14C.

### DETAILED DESCRIPTION

The following description of the embodiments of the invention is not intended to limit the invention to these embodiments, but rather to enable any person skilled in the art to make and use this invention.

As used herein, “sequence” preferably refers to a nucleotide sequence, such as a DNA sequence, an RNA sequence, or other sequences of nucleotides, but can also refer to an amino acid sequence, or any other biological sequence of repeating units. Nucleic acids according to embodiments can have any suitable form, including linear, circular, and any suitable strandedness, including single stranded, and double stranded, for any particular host species and/or purpose. Also, nucleic acids according to examples can be incorporated into genetic constructs according to examples, as described in detail below. Also, while DNA sequences are described herein, it is understood that corresponding RNA sequences are contemplated by the inventors and are considered within the scope of the invention. RNA sequences may have individual utility beyond the utility of DNA sequences.

Nucleic acids according to embodiments can be synthesized using any suitable techniques, procedures, and processes, such as solid-phase synthesis, enzymatic synthesis, such as reverse transcription using reverse transcriptase to generate complementary DNA (cDNA) from an RNA template. Polymerase chain reaction (PCR) and polymerase cycling assembly (PCA) techniques can also be used in synthesis of nucleic acids according to embodiments.

Once synthesized, nucleic acids according to embodiments can be further processed and/or treated using one or more suitable techniques, procedures, and processes for purification, quality assessment, and quantification. For example, nucleic acids according to embodiments can be subjected to high-performance liquid chromatography (HPLC), ultrafiltration, size-exclusion chromatography, or other suitable techniques for removing contaminants. Quality and quantity assessments can be conducted using spectroscopy, electrophoresis, or other analytical methods to ensure integrity and consistency of the synthesized nucleic acids according to embodiments.

As used herein, “activator” can optionally refer to the activator sequence (e.g., the sequence of the activator). As used herein, “repressor” can optionally refer to the repressor sequence (e.g., the sequence of the repressor).

As used herein, the term “percent identity” refers to a percentage of identity between two referenced nucleotide sequences. The percent identity of two nucleotide sequences is determined by aligning the referenced sequences for optimal comparison (e.g., introducing gaps if necessary). The percent identity is calculated as follows:

$$\% identity = ( \# of identical positions / total \# of positions ) \times 100$$

where an identical position is one in which the nucleotide in one sequence is the same as the nucleotide in the corresponding position of the other sequence. A non-limiting example of a sequence comparison algorithm is described in Karlin et al., Proc. Natl. Acad. Sci. USA 90:5873-5877 (1993). This algorithm is implemented in the NBLAST and XBLAST programs (version 2.0), as described in Altschul et al., Nucleic Acids Res. 25:389-3402 (1997). Unless otherwise specified, the default parameters of the respective BLAST programs (e.g., NBLAST) may be used for sequence comparison. In some embodiments, parameters may be set at a score threshold of 100 and a word length of 12, with optional variations such as a word length of 5 or 20.

As used herein, the term “plurality” refers to a quantity of at least two members. In some embodiments, a plurality may comprise 5 or more, 10 or more, 50 or more, 100 or more, 1000 or more, 10,000 or more, or up to 100,000 or more members.

Where a range of values is provided, it is understood that each intervening value within the stated range—including values to the nearest tenth of the unit of the lower limit, unless the context clearly dictates otherwise—is encompassed within the described range. The upper and lower limits of the range, as well as any intermediate values, are included within the scope of the disclosure unless explicitly stated otherwise. Additionally, where a range includes one or both of its endpoints, variations excluding either or both endpoints are also encompassed within the disclosure.

Unless otherwise defined, all technical and scientific terms used herein have the same meaning as commonly understood by those of ordinary skill in the relevant art. While methods and systems similar or functionally equivalent to those described herein may be used in the practice or testing of the disclosed methods and systems, representative and illustrative embodiments are described below.

For clarity, certain features of the disclosed systems, methods, and compositions are described separately in distinct examples. However, it is understood that these features may also be combined into a single example. Conversely, features that are described together in a single example may also be provided separately or in any operable sub-combination. All combinations and sub-combinations of the disclosed examples are expressly included within the scope of the present disclosure, provided they result in an operable system, method, or composition.

As will be apparent to those skilled in the art upon reading this disclosure, each of the examples described herein comprises distinct components, features, and/or steps that may be separated or combined without departing from the scope or spirit of the invention. Any described method may be carried out in the order of steps explicitly recited or in any logically permissible order unless otherwise stated.

### 1. Overview

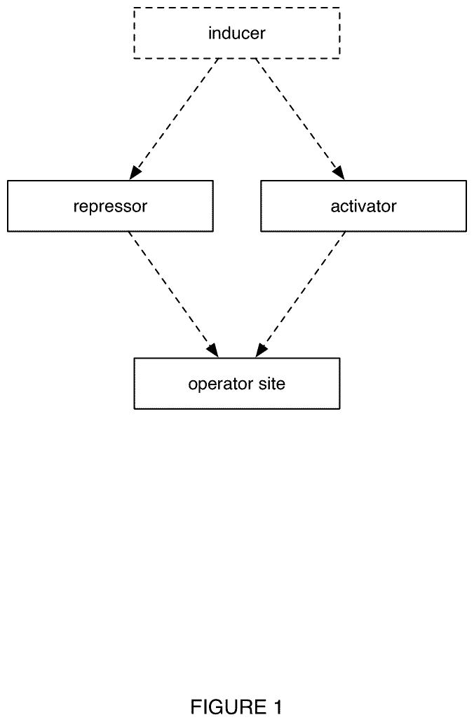

As shown in FIG. 1, the inducible promoter system can include an activator, a repressor, and an operator site. However, the inducible promoter system can additionally or alternatively include any other elements.

In variants, the inducible promoter system can function to control expression of one or more genes of interest in a cell.

### 2. Examples

In variants, the inducible promoter system can include a reverse cumate transcriptional activator (e.g., transactivator) (rcTA) as well as a cumate repressor. The cumate repressor can be a wild-type cumate repressor (CymR) or a genetically modified cumate repressor. In an example, the cumate repressor can be a cumate transcriptional silencer (cTS) (e.g., a fusion protein containing a cumate repressor fused to a silencer domain).

In a first example, the sequence for the rcTA and the sequence for the cumate repressor contain matching CymR sequences (e.g., corresponding to a DNA binding domain, cumate binding domain, and dimerization domain of the CymR), derived from the same bacteria species and/or strains. In a second example, the sequence for the rcTA and the sequence for the cumate repressor contain differing CymR sequences, derived from differing bacteria species and/or strains. In a specific example, the sequence for the rcTA contains a first CymR sequence derived from a first bacteria species and/or strain, while the sequence for the cumate repressor contains a second CymR sequence derived from a second bacteria species and/or strain (e.g., where a portion of the second CymR sequence differs from the first CymR sequence). In a third example, the rcTA and the cumate repressor contain differing dimerization domains (e.g., derived from different bacteria species and/or strains), but the same DNA binding domain. In a first specific example, the sequence for the cumate repressor contains a hybrid CymR sequence derived from two different bacteria species and/or strains. In a second specific example, the sequence for the rcTA contains a hybrid CymR sequence derived from two different bacteria species and/or strains.

### 3. Technical Advantages

Variants of the technology can confer one or more advantages over conventional technologies.

First, many inducible promoter systems “leak” (e.g., transcription still occurs despite an absence of the inducer). Variants of the technology can generate an inducible promoter system with reduced leakiness. For example, the inducible promoter system can reduce leakiness by including both a reverse cumate transcriptional activator (rcTA) as well as a cumate repressor (e.g., a wild-type cumate repressor or a genetically modified cumate repressor), where the rcTA activates transcription in the presence of cumate and the cumate repressor represses transcription in the absence of cumate. In a specific example, the cumate repressor can be a fusion protein containing a cumate repressor fused to all or a portion of another transcriptional repressor (e.g., the kid-1 protein); this can improve the efficacy of the cumate repressor, further reducing leakiness. In another specific example, the sequence for the rcTA can contain CymR sequences from a first bacteria species, and the sequence for the cumate repressor can contain CymR sequences from a second, different bacteria species (or a second strain within the same bacteria species), resulting in the rcTA and the cumate repressor containing different dimerization domains (and, optionally, differing DNA binding domains); this can reduce dimerization between the rcTA and the cumate repressor. In another specific example, the sequence for the cumate repressor can contain CymR sequences from different bacteria species and/or different strains within a species, resulting in the rcTA and the cumate repressor containing the same DNA binding domains and different dimerization domains; this can reduce dimerization between the rcTA and the cumate repressor without negatively impacting DNA binding for either protein.

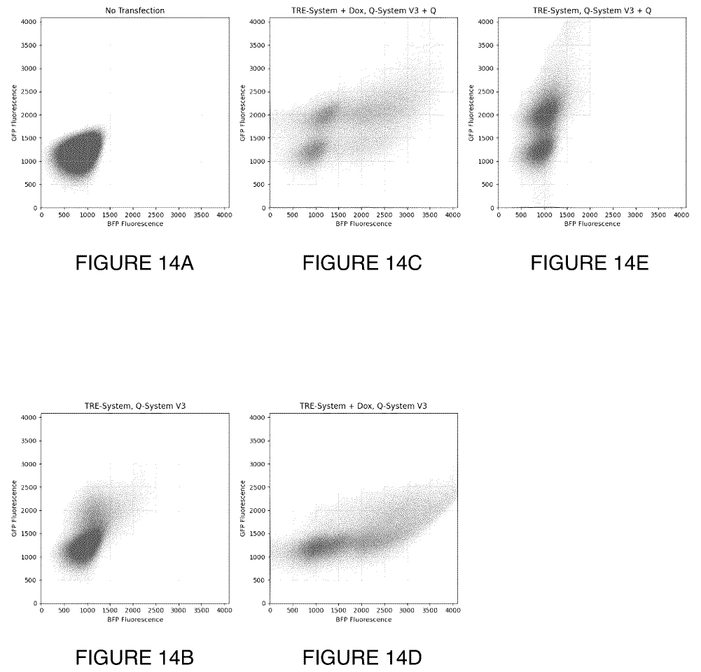

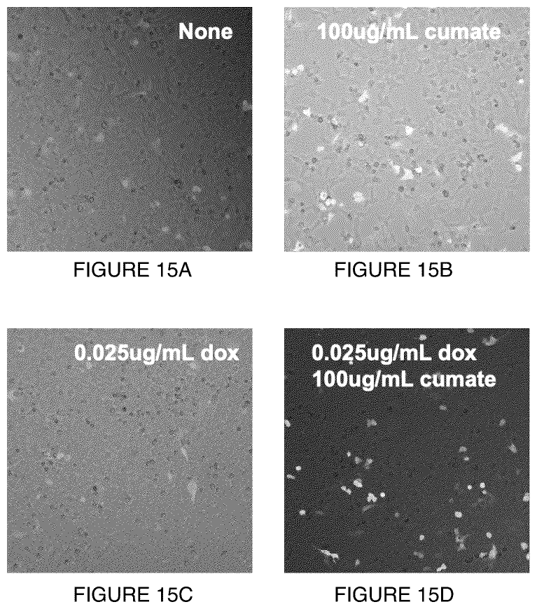

Second, variants of the technology can enable two inducible promoter systems to be used independently. In a specific example, a cumate-inducible promoter subsystem can be orthogonal to a doxycycline-inducible promoter subsystem (e.g., a Tetracycline Response Element (TRE) system), where there is limited or no interaction between cumate and components (e.g., activator and/or repressor) of the doxycycline-inducible promoter subsystem, and limited or no interaction between doxycycline and components (e.g., activator and/or repressor) of the cumate-inducible promoter subsystem. In variants, this orthogonality can enable independent regulation of two different genes of interest within the same cell or across different cells (e.g., different cells exposed to the same media). Examples are shown in FIG. 14A, FIG. 14B, FIG. 14C, FIG. 14D, FIG. 14E, FIG. 15A, FIG. 15B, FIG. 15C, and FIG. 15D.

However, further advantages can be provided by the systems, methods, and compositions disclosed herein.

### 4. Inducible Promoter System

As shown in FIG. 1, the inducible promoter system can include an activator, a repressor, and an operator site. The inducible promoter system can optionally include an inducer, a regulatory element, a gene of interest, and/or any other suitable elements.

The activator functions to increase transcription of a gene of interest (e.g., in the presence of the inducer). The activator is preferably controlled based on the presence (e.g., concentration) of the inducer, but can alternatively not be controlled based on the presence of the inducer. The activator preferably binds to the operator site (activating transcription) in the presence of the inducer (e.g., when the activator is bound to one or more copies of the inducer). However, the activator can alternatively bind to the operator site in the absence of the inducer (e.g., when the activator is not bound to one or more copies of the inducer).

In a first example, the activator can be an engineered composition including an engineered protein (e.g., translated from an engineered sequence). In a second example, the activator (e.g., the activator sequence) can be an engineered composition including an engineered sequence (e.g., DNA sequence).

One or more sequences (e.g., subsequences) within the activator sequence can optionally be derived from and/or substantially equivalent to all or a portion of a cumate repressor CymR sequence. The CymR sequence (e.g., wild type CymR sequence) can optionally be found in one or more bacteria (e.g., a bacteria strain and/or species), such as: Pseudomonas putida strain KL47, Pseudomonas putida strain PL-W, Pseudomonas putida strain JT101, Pseudomonas putida strain JT810, Pseudomonas putida strain F1, Rhodococcus sp. T104, Rhodopseudomonas palustris, Paraburkholderia xenovorans strain LB400, and/or any other organism using cymene and/or cumate. In an example, a sequence within the activator sequence can correspond to one or more domains within a CymR sequence (e.g., DNA binding domain, dimerization domain, cumate binding domain, portions thereof, etc.).

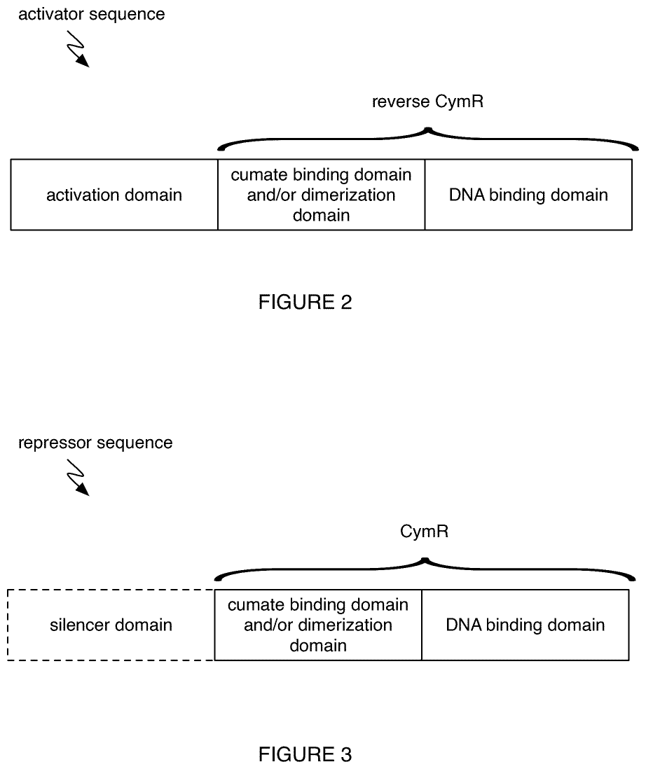

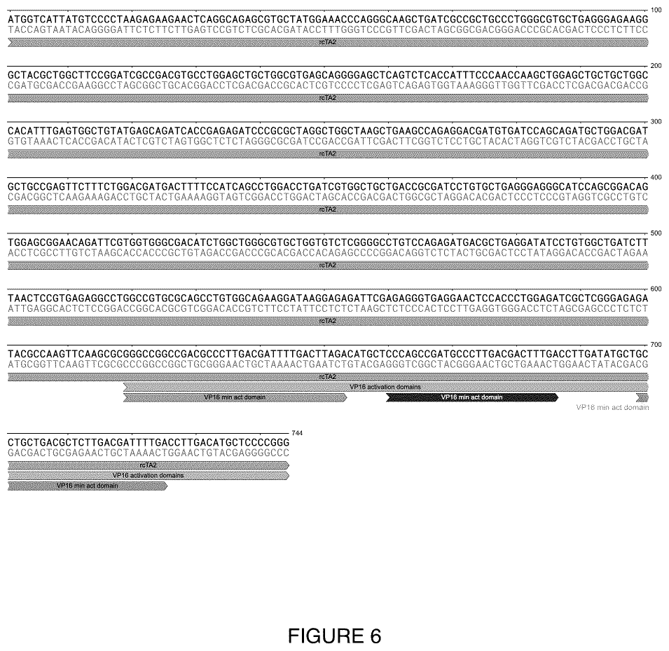

In variants, the activator can include or be based on a reverse cumate repressor (e.g., reverse CymR). An example is shown in FIG. 2. For example, the activator can include a reverse cumate transcriptional activator (rcTA), a fusion protein containing a reverse CymR (a mutated CymR, which binds to the operator site in the presence of cumate) and an activation domain (e.g., a VP16 activation domain). In a first example, the sequence of the reverse CymR portion of the activator can be derived from and/or substantially equivalent to a CymR sequence from a single bacteria species and/or strain therein (e.g., Pseudomonas putida strain F1, Paraburkholderia xenovorans strain LB400, etc.). An example nucleic acid that encodes a suitable activator is set forth in SEQ ID NO: 1, and is also illustrated in FIG. 6. In a second example, the sequence of the reverse CymR portion of the activator can be a hybrid, derived from and/or substantially equivalent to a CymR sequence from two or more bacteria species and/or strains therein. In a specific example a first domain (e.g., the DNA binding domain) of the reverse CymR can be derived from and/or substantially equivalent to a first bacteria species and/or strain, and a second domain (e.g., the dimerization domain, cumate binding domain, etc.) of the reverse CymR can be derived from and/or substantially equivalent to a second bacteria species and/or strain.

However, the activator can be otherwise configured.

The repressor functions to decrease transcription of a gene of interest (e.g., in the presence of the inducer). The repressor is preferably controlled based on the presence (e.g., concentration) of the inducer, but can alternatively not be controlled based on the presence of the inducer. The repressor preferably binds to the operator site (repressing transcription) in the absence of the inducer (e.g., when the repressor is not bound to one or more copies of the inducer). However, the repressor can alternatively bind to the operator site in the presence of the inducer (e.g., when the activator is bound to one or more copies of the inducer).

In a first example, the repressor can be an engineered composition including an engineered protein (e.g., translated from an engineered sequence). In a second example, the repressor (e.g., the repressor sequence) can be an engineered composition including an engineered sequence (e.g., DNA sequence).

One or more sequences (e.g., subsequences) within the repressor sequence can optionally be derived from and/or substantially equivalent to all or a portion of a cumate repressor CymR sequence. The CymR sequence (e.g., wild type CymR sequence) can optionally be found in one or more bacteria (e.g., a bacteria strain and/or species), such as: Pseudomonas putida strain KL47, Pseudomonas putida strain PL-W, Pseudomonas putida strain JT101, Pseudomonas putida strain JT810, Pseudomonas putida strain F1, Rhodococcus sp. T104, Rhodopseudomonas palustris, Paraburkholderia xenovorans strain LB400, and/or any other organism using cymene and/or cumate. In an example, a sequence within the repressor sequence can correspond to one or more domains within a CymR sequence (e.g., DNA binding domain, dimerization domain, cumate binding domain, portions thereof, etc.).

In variants, the repressor can include or be based on a cumate repressor (e.g., CymR). An example is shown in FIG. 3. In a first example, the sequence of the CymR portion of the repressor can be derived from and/or substantially equivalent to a CymR sequence from a single bacteria species and/or strain therein (e.g., Pseudomonas putida strain F1, Paraburkholderia xenovorans strain LB400, etc.). In a second example, the sequence of the CymR portion of the repressor can be a hybrid, derived from and/or substantially equivalent to a CymR sequence from two or more bacteria species and/or strains therein. In a specific example a first domain (e.g., the DNA binding domain) of the CymR portion of the repressor can be derived from and/or substantially equivalent to a first bacteria species and/or strain, and a second domain (e.g., the dimerization domain, cumate binding domain, etc.) of the CymR portion of the repressor can be derived from and/or substantially equivalent to a second bacteria species and/or strain.

The repressor can optionally include a fusion protein containing a cumate repressor. In a specific example, the repressor can be a cumate transcriptional silencer (cTS) (e.g., a fusion protein containing a cumate repressor and a silencer domain). In an illustrative example, the repressor can be a fusion between CymR, all or a portion of a repressor and/or silencer element (e.g., kid-1), and/or a nuclear localization signal (e.g., SV40 NLS).

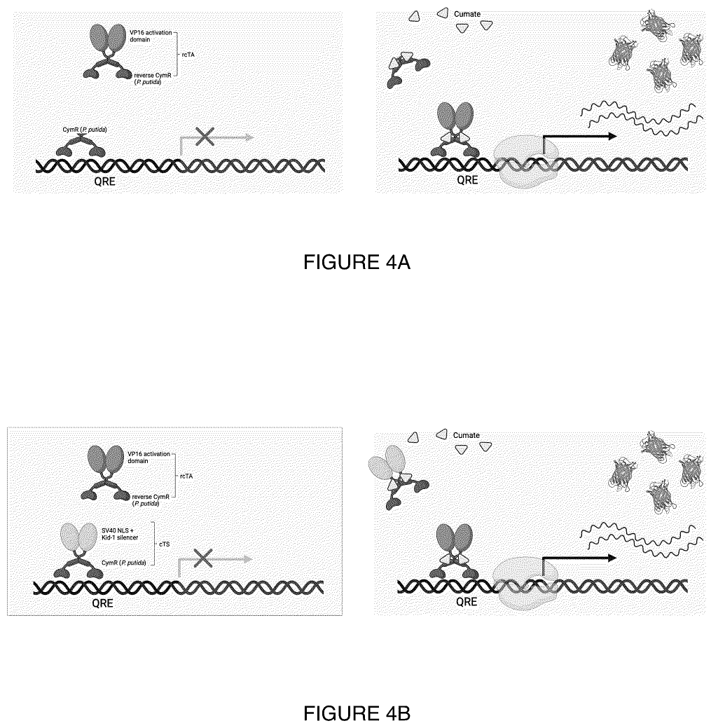

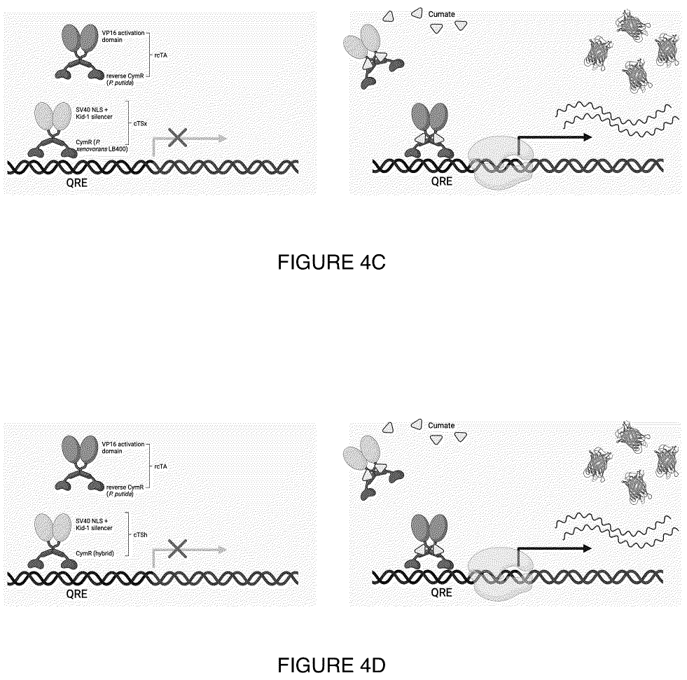

In a first embodiment, the sequences of the activator and the repressor contain matching CymR sequences (e.g., DNA binding domain, cumate binding domain, and dimerization domain), derived from and/or substantially equivalent to the same bacteria species and/or strains. Examples are shown in FIG. 4A and FIG. 4B. In a second embodiment, the sequences of the activator and the repressor contain differing CymR sequences. In an example, the sequence of the activator contains a first CymR sequence derived from and/or substantially equivalent to a first bacteria species and/or strain, while the sequence of the repressor contains a second CymR sequence derived from and/or substantially equivalent to a second bacteria species and/or strain. In a specific example, a portion of the second CymR sequence (e.g., corresponding to one or more of: DNA binding domain, cumate binding domain, dimerization domain, and/or portions thereof) differs from the first CymR sequence. An example is shown in FIG. 4C. In a third embodiment, the activator and the repressor have differing dimerization domains, and the same DNA binding domain. An example is shown in FIG. 4D. In a first specific example, the sequence of the repressor contains a hybrid CymR sequence derived from two different bacteria species and/or strains. In a second specific example, the sequence of the activator contains a hybrid CymR sequence derived from two different bacteria species and/or strains.

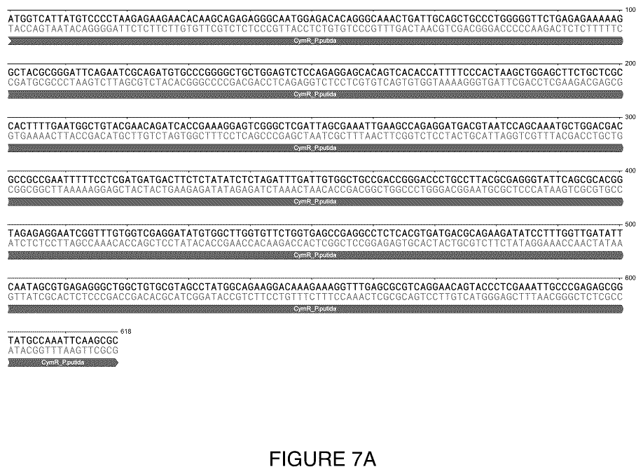

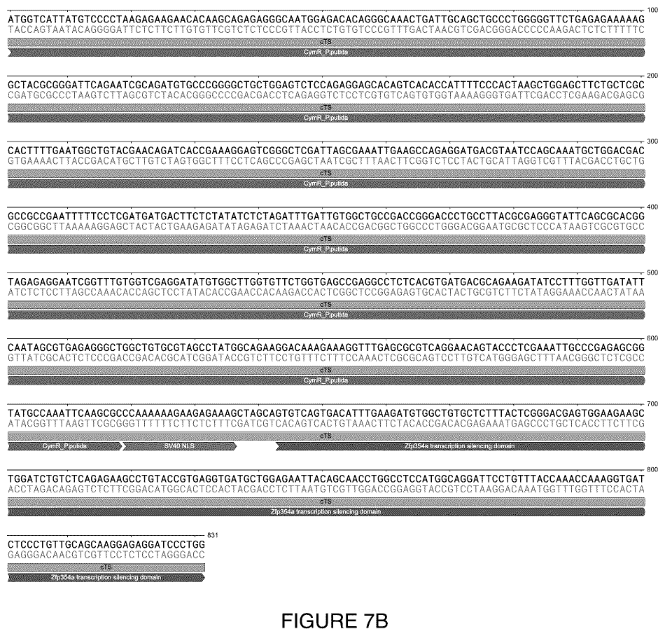

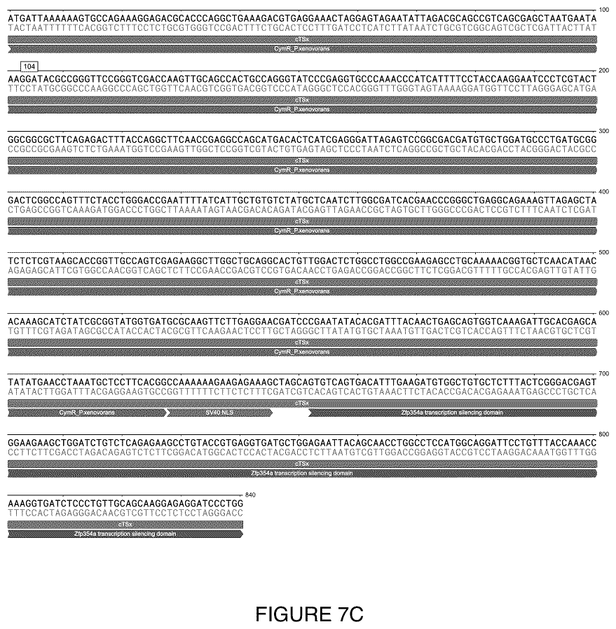

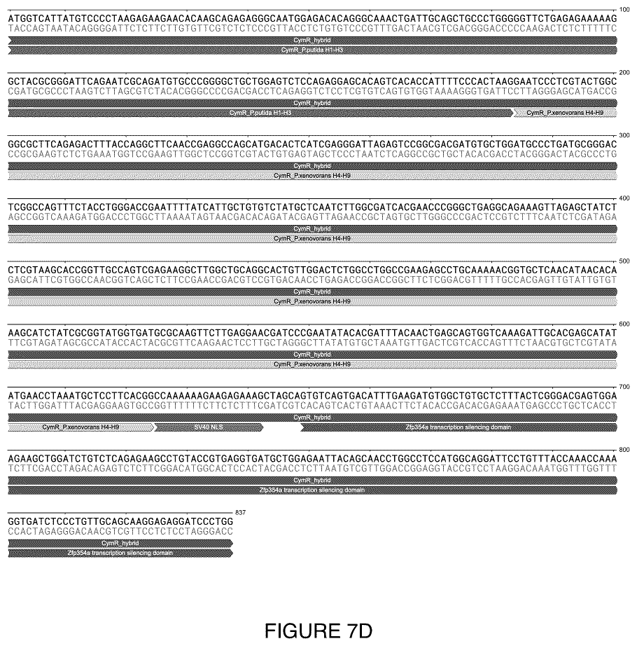

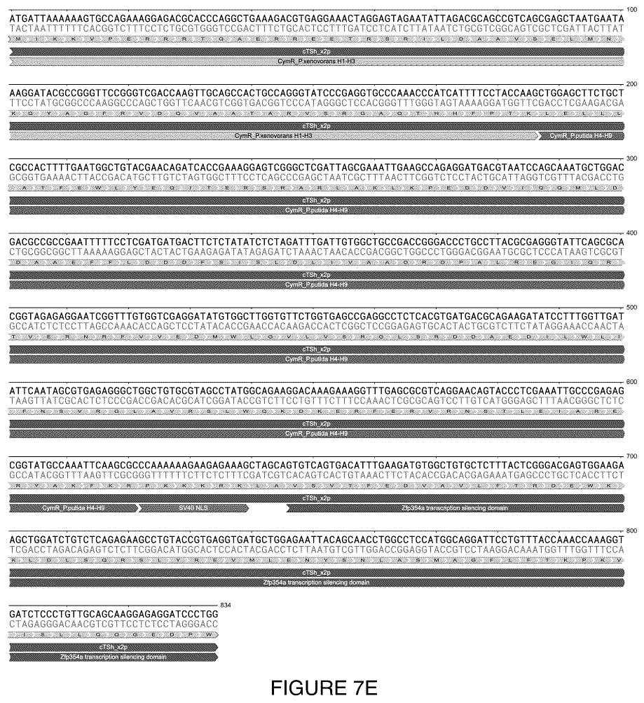

An example nucleic acid that encodes a suitable repressor is set forth in SEQ ID NO: 2, and is also illustrated in FIG. 7A. Another example nucleic acid that encodes a suitable repressor is set forth in SEQ ID NO: 3, and is also illustrated in FIG. 7B. Another example nucleic acid that encodes a suitable repressor is set forth in SEQ ID NO: 4, and is also illustrated in FIG. 7C. Another example nucleic acid that encodes a suitable repressor is set forth in SEQ ID NO: 5, and is also illustrated in FIG. 7D. Another example nucleic acid that encodes a suitable repressor is set forth in SEQ ID NO: 6, and is also illustrated in FIG. 7E.

However, the repressor can be otherwise configured.

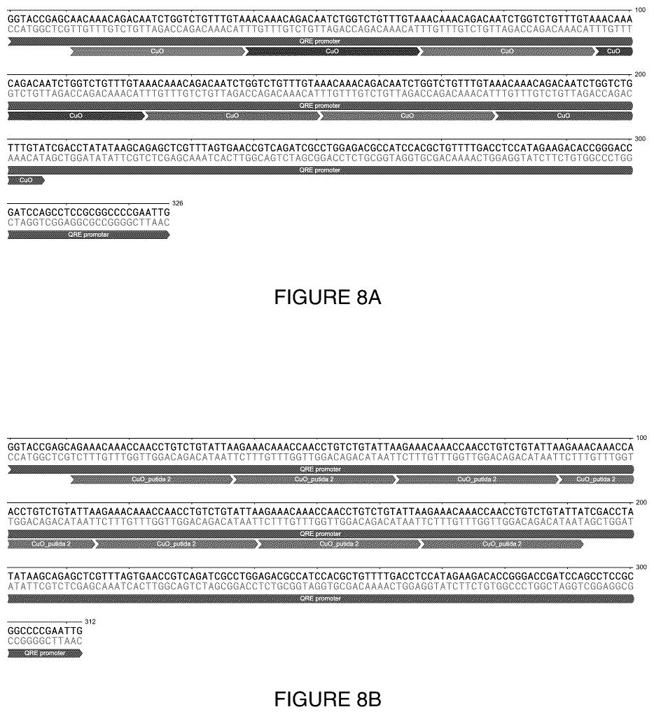

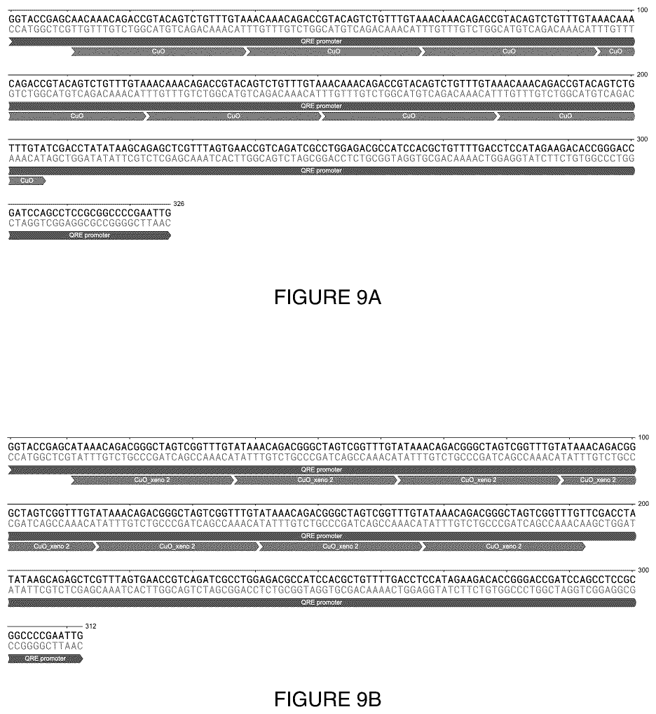

The operator site functions to provide a binding site for the activator and/or repressor to regulate expression of the gene of interest. In a specific example, both the activator and the repressor bind to the operator site (e.g., as controlled by the inducer). In an illustrative example, the activator binds to the operator site in the presence of the inducer (e.g., when one or more cumate molecules bind to the activator) and the repressor binds to the operator site in the absence of the inducer (e.g., when one or more cumate molecules are not bound to the repressor). The operator site can include the regulatory element, be located within the regulatory element, bound the regulatory element, be adjacent to the regulatory element, be upstream of the regulatory element, and/or be otherwise located relative to the operator site. The operator site is preferably upstream of the gene of interest, but can alternatively be otherwise located relative to the gene of interest. In an example, the operator site can include one or more copies (e.g., 2, 3, 4, 5, 6, 7, 8, 9, 10, etc.) of an operator sequence (e.g., CuO). The (repeated) operator sequence can optionally be derived from and/or be substantially equivalent to operator sequences found in organisms using cymene and/or cumate (e.g., a previously listed bacteria). In a specific example, the operator site can include a sequence shown in FIG. 8A, FIG. 8B, FIG. 9A, and/or FIG. 9B. An example nucleic acid sequence that encodes a suitable operator site is set forth in SEQ ID NO: 7, and is also illustrated in FIG. 8A. Another example nucleic acid sequence that encodes a suitable operator site is set forth in SEQ ID NO: 8, and is also illustrated in FIG. 8B. Another example nucleic acid sequence that encodes a suitable operator site is set forth in SEQ ID NO: 9, and is also illustrated in FIG. 9A. Another example nucleic acid sequence that encodes a suitable operator site is set forth in SEQ ID NO: 10, and is also illustrated in FIG. 9B. However, the operator site can be otherwise configured.

The inducible promoter system can optionally include or interface with a regulatory element, which functions to regulate expression of the gene of interest. The regulatory element can include the operator site, be within the operator site, be downstream of the operator site, and/or be otherwise located relative to the operator site. The regulatory element is preferably upstream of the gene of interest, but can alternatively be otherwise located relative to the gene of interest. The regulatory element is preferably a promoter, but can additionally or alternatively include an enhancer, a silencer, and/or any other transcriptional regulatory element. In a specific example, the regulatory element can include a cumate responsive element (QRE) promoter. In a specific example, the regulatory element can include a sequence set forth in SEQ ID NO: 7, a sequence set forth in SEQ ID NO: 8, a sequence set forth in SEQ ID NO: 9, and/or a sequence set forth in SEQ ID NO: 10. However, the regulatory element can be otherwise configured.

The inducible promoter system can optionally include or interface with a gene of interest. A gene of interest is one or more expressible genes (i.e., one or more genes capable of undergoing transcription to create RNA, such as messenger RNA, suitable for translation into an amino acid sequence, such as a protein). A gene of interest can include one or more genes. Examples of a gene of interest and/or genes therein can include: a selectable marker (e.g., selection marker), kill switch gene (e.g., suicide gene), transcription factor, opsin, cell differentiation genes, cell adhesion molecule genes, hypoimmunogenicity genes, fluorophore genes, cell surface molecule expression genes, one or more attachment sites, a landing site, any transgene, cell programming genes (e.g., to induce differentiation) multiple genes (in sequence), and/or any other DNA sequence. However, the gene of interest can be otherwise configured.

The inducible promoter system can optionally include and/or interface with one or more inducers. The inducer functions to regulate activity of the activator and/or the repressor (e.g., to regulate expression of a gene of interest). For example, the inducer can regulate the activity of the activator and the activity of the repressor. In a specific example, the inducer can increase (e.g., enable) binding of the activator to the operator site (e.g., downstream of the regulatory element, a component of the regulatory element, etc.) and can decrease (e.g., disable) binding of the repressor to the operator site. The inducer is preferably a small molecule, but can additionally or alternatively include any other inducer. In a first example, the inducer is cumate. In a second example, the inducer is doxycycline. However, the inducer can be otherwise configured.

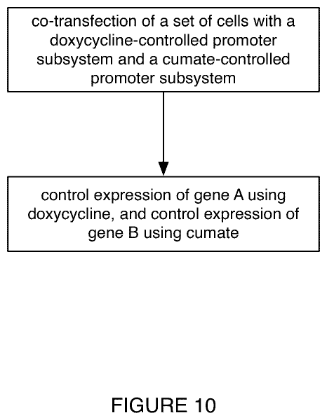

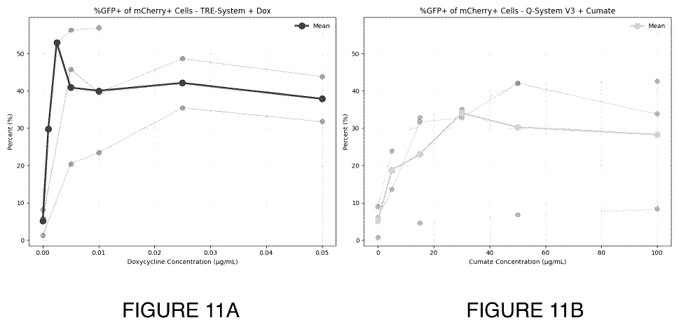

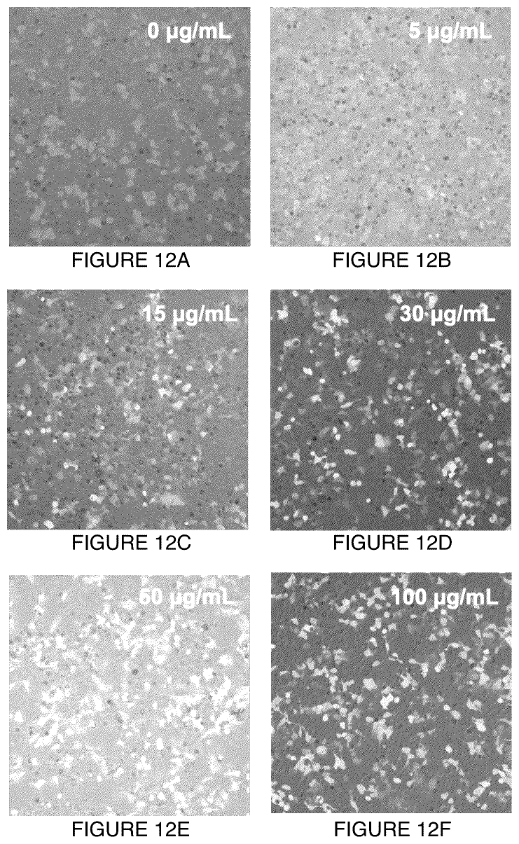

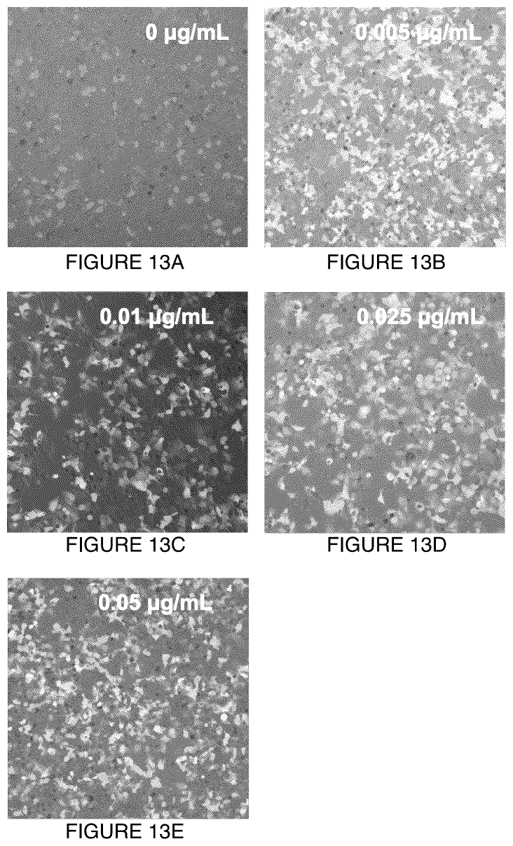

In a first variant, the inducible promoter system interfaces with a single inducer to regulate expression of a single gene of interest. In a second variant, the inducible promoter system interfaces with two inducers (e.g., cumate and doxycycline) to regulate expression of two genes of interest. An example is shown in FIG. 10. For example, the inducible promoter system can include: a first inducible promoter subsystem (including an activator and/or a repressor) regulating expression of a first gene of interest based on the concentration of a first inducer (e.g., cumate) in the cell; and a second inducible promoter subsystem (including an activator and/or a repressor) regulating expression of a second gene of interest based on the concentration of a second inducer (e.g., doxycycline) in the cell. Examples are shown in FIG. 11A, FIG. 11B, FIGS. 12A-12F, and FIGS. 13A-13E. In a specific example, the first inducible promoter subsystem can be orthogonal to the second inducible promoter subsystem, where there is limited or no interaction between the first inducer and components (e.g., activator and/or repressor) of the second inducible promoter subsystem, and there is limited or no interaction between the second inducer and components (e.g., activator and/or repressor) of the first inducible promoter subsystem. Examples are shown in FIGS. 14A-14D and FIGS. 15A-15D. In variants, this orthogonality can enable independent regulation of the first and second gene of interest. In a specific example, expression of the first gene of interest and the second gene of interest can be induced (e.g., activated) at different times. An example nucleotide sequence that encodes a suitable transcription activator for the second inducible promoter subsystem is set forth in SEQ ID NO: 11. An example nucleotide sequence that encodes a suitable transcription repressor for the second inducible promoter subsystem is set forth in SEQ ID NO: 12. An example nucleotide sequence that encodes a TRE promoter, which is an example of a suitable operator site for the second inducible promoter subsystem, is set forth in SEQ ID NO: 13.

In an example, the first inducible promoter subsystem is a cumate-inducible promoter system controlling expression of a first gene of interest, and the second inducible promoter subsystem is a doxycycline-inducible promoter subsystem (e.g., a TRE system) controlling expression of a second gene of interest. In a specific example, when a cell includes the cumate-inducible promoter subsystem and the doxycycline-inducible promoter subsystem (e.g., the cell is co-transfected with a first plasmid containing the cumate-inducible promoter subsystem and a second plasmid containing the doxycycline-inducible promoter subsystem), increasing a concentration of cumate in the cell and/or the cell media increases expression of the first gene of interest by a greater percentage than the second gene of interest. In another specific example, when a cell includes the cumate-inducible promoter subsystem and the doxycycline-inducible promoter subsystem, increasing a concentration of doxycycline in the cell and/or the cell media increases expression of the second gene of interest by a greater percentage than the first gene of interest. In another specific example, when a set of cells include the cumate-inducible promoter subsystem and the doxycycline-inducible promoter subsystem, increasing a concentration of cumate in the set of cells and/or the cell media from 0 μg/mL to 100 ug/mL increases expression of the first gene of interest (on average, across the set of cells) by at least 25 percent (e.g., at least 30%, at least 40%, at least 45%, etc.) and increases expression of the second gene of interest (on average, across the set of cells) by less than 25 percent (e.g., less than 20%, less than 15%, less than 10%, etc.); an example is shown in FIG. 14E. In another specific example, when a set of cells include the cumate-inducible promoter subsystem and the doxycycline-inducible promoter subsystem, increasing a concentration of doxycycline in the set of cells and/or the cell media from 0 μg/mL to .025 μg/mL increases expression of the second gene of interest (on average, across the set of cells) by at least 25 percent (e.g., at least 30%, at least 40%, at least 45%, at least 50%, etc.) and increases expression of the first gene of interest (on average, across the set of cells) by less than 25 percent (e.g., less than 20%, less than 15%, less than 10%, etc.); an example is shown in FIG. 14D.

In a first embodiment of the second variant, the inducible promoter system includes: a first inducible promoter subsystem regulating expression of a first gene of interest in a genome of a cell based on the concentration of a first inducer (e.g., cumate) in the cell; and a second inducible promoter subsystem regulating expression of a second gene of interest in the genome of the cell based on the concentration of a second inducer (e.g., doxycycline) in the cell. In an example, the first xxx is a cumate-inducible promoter system, and the second inducible promoter subsystem is a doxycycline-inducible promoter system. In a specific example, the doxycycline-inducible promoter system is a Tetracycline Response Element (TRE) system. In an example, the first gene of interest and the second gene of interest can be independently regulated within the same cell. In an illustrative example, the first gene of interest and the second gene of interest can be different cell programming genes, wherein expression of the first gene of interest is induced at different times relative to the second gene of interest. In another illustrative example, the first gene of interest is an excitatory opsin and the second gene of interest in an inhibitory opsin (e.g., enabling temporally dependent optogenetic response). In another illustrative example, the first gene of interest and the second gene of interest can control release of different small molecules. In another illustrative example, the first gene of interest and the second gene of interest can be used for chemogenetic signaling.

In a second embodiment of the second variant, the inducible promoter system includes: a first inducible promoter subsystem regulating expression of a first gene of interest in a genome of a first cell based on the concentration of a first inducer (e.g., cumate) in the first cell; and a second inducible promoter subsystem regulating expression of a second gene of interest in a genome of a second cell based on the concentration of a second inducer (e.g., doxycycline) in the second cell. In an illustrative example, the first gene of interest and the second gene of interest can be independently regulated across different cells exposed to a common environment (e.g., shared media). In a specific example, the first cell and the second cell have about the same concentration of the first inducer (e.g., cumate) and/or about the same concentration of the second inducer (e.g., doxycycline). In another specific example, the first cell and the second cell are exposed to the same media. In another specific example, the first cell has a different genome than the second cell. In another specific example, a first operator site (e.g., designed to interface with the activator and/or repressor of the first inducible promoter subsystem) is integrated into the first cell and a second operator site (e.g., designed to interface with the activator and/or repressor of the second inducible promoter subsystem) is integrated into the second cell. In another specific example, a first operator site is integrated into the first cell and is not integrated into the second cell. In another specific example, a second operator site is integrated into the second cell and is not integrated into the first cell. In another specific example, a combination of the previous specific examples can be implemented. However, the inducible promoter system can otherwise interface with one or more inducers.

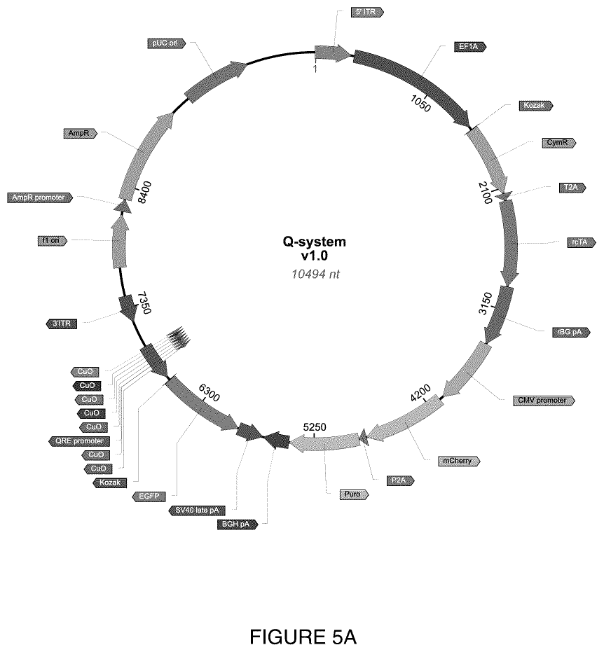

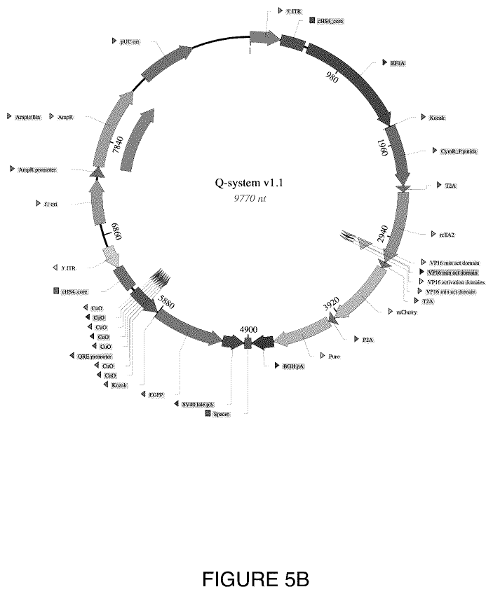

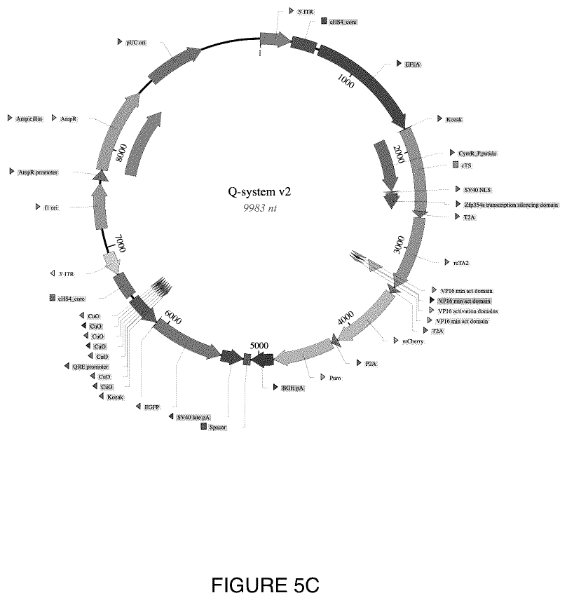

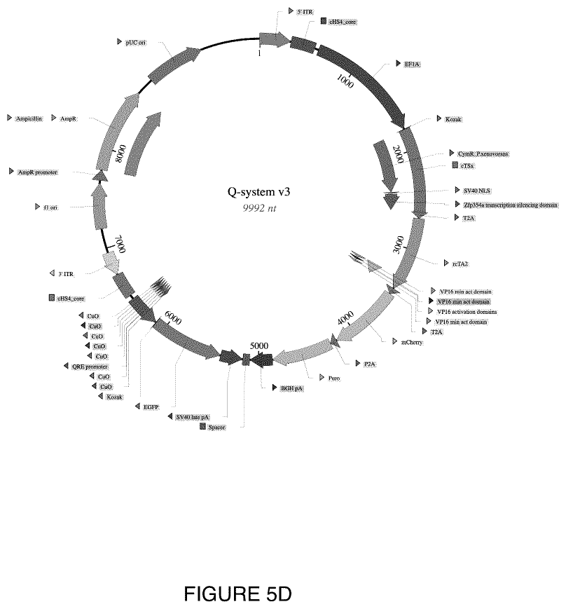

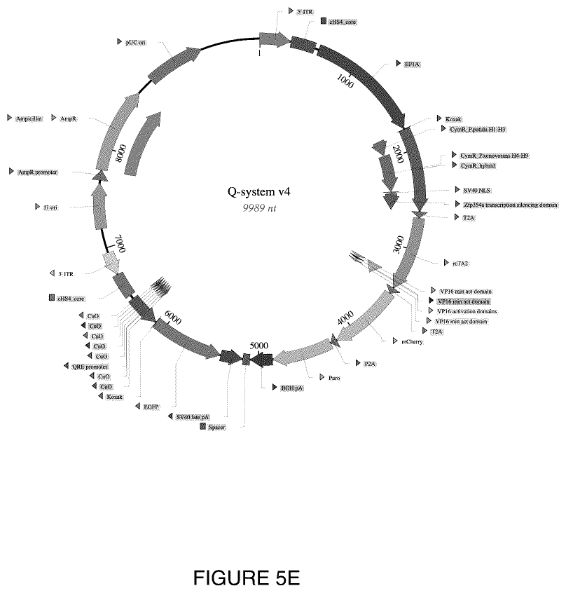

The inducible promoter system can optionally include one or more sequences (e.g., genetically engineered sequences) for the activator, the repressor, the operator site, the regulatory element, the gene of interest, and/or any other component of the inducible promoter system. The inducible promoter system can optionally include one or more vectors (e.g., plasmids, constructs, etc.) containing sequences for the activator, the repressor, the operator site, the regulatory element, the gene of interest, and/or any other component of the inducible promoter system. An example nucleotide sequence for a suitable plasmid is set forth in SEQ ID NO: 14 and includes the transcription activator set forth in SEQ ID NO: 11, the transcription suppressor set forth in SEQ ID NO: 12, and the TRE promoter set forth in SEQ ID NO: 13. The plasmid is suitable for use as a vector containing sequences for an inducible promoter system, or inducible promoter subsystem, responsive to doxycycline. The plasmid can be used with another vector according to an embodiment, such as a plasmid, containing sequences for an inducible promoter system, or inducible promoter subsystem, responsive to a different activator, such as cumate, as described herein. Specific examples of such vectors are shown in FIG. 5A, FIG. 5B, FIG. 5C, FIG. 5D, and FIG. 5E. Nucleotide sequences for these example vectors are set forth in SEQ ID NO: 15, SEQ ID NO: 16, SEQ ID NO: 17, SEQ ID NO: 18, and SEQ ID NO: 19, respectively. For two vector systems, both vectors can be contained within the same cell. Alternatively, the vectors of such two vector systems can be contained in different cells. The inducible promoter system can optionally include one or more cell genomes (e.g., genetically engineered cell genomes) containing sequences for the activator, the repressor, the operator site, the regulatory element, the gene of interest, and/or any other component of the inducible promoter system.

In specific examples, the inducible promoter system includes one or more of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and/or SEQ ID NO: 19. In some cases, the inducible promoter system includes a nucleotide sequence that differs by 1, 2, 3, 4, or 5 nucleotides from any of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and/or SEQ ID NO: 19.

The inducible promoter system can optionally include one or more cells containing the activator, the repressor, the operator site, the regulatory element, the gene of interest, sequences thereof, and/or any other component of the inducible promoter system. The cell can be a human cell, any other animal cell, a bacteria cell, and/or any other cell. The cell is preferably a stem cell (e.g., an induced pluripotent stem cell), but can alternatively not be a stem cell. The cell can optionally be a genetically engineered cell. The cell can optionally be a part of a cell line, be used to produce a cell line (e.g., the cell is a parent cell for a cell line), and/or otherwise used.

The method can optionally include: transfecting a cell with one or more vectors, integrating one or more sequences (e.g., the operator site, the regulatory element, the gene of interest, the activator sequence, the repressor sequence, etc.) into a cell genome, controlling inducer concentration, and/or any other suitable steps. All or portions of the method can use recombinase technology (e.g., site-specific recombinase technology), prime editing, homology-directed repair (e.g., homologous recombination), any CRISPR technology, and/or any other genetic engineering method. All or portions of the method can be repeated for one or more inducible promoter systems and/or subsystems thereof. All or portions of the method can be performed iteratively, concurrently, asynchronously, periodically, simultaneously, in parallel, in series, and/or at any other suitable time. Methods described herein may include multiple steps performed sequentially or with an intervening delay. An interval between steps may range from immediate execution upon completion of the preceding step to predefined waiting periods. In some examples, the interval between steps is at least 1 second, 10 seconds, 30 seconds, 60 seconds, 5 minutes, 10 minutes, 60 minutes, or 5 hours. In other embodiments, subsequent steps are performed immediately after the completion of a prior step or after an incubation or waiting period of a few minutes to overnight. However, the inducible promoter system can be otherwise configured.

It is contemplated that the sequences disclosed herein may tolerate minor variations while retaining their functional properties. Such variations may include conservative substitutions, insertions, deletions, or modifications that do not materially alter the structure, stability, or activity of the encoded nucleic acid or protein. Accordingly, sequences having at least 70%, 75%, 80%, 85%, 90%, 91%, 92%, 93%, 94%, 95%, 96%, 97%, 98%, or 99% identity to any of the disclosed sequences are considered within the scope of the invention. Sequence percent identity may be determined using standard alignment algorithms such as BLAST, ClustalW, or other suitable computational methods known in the art. Variants that retain substantially equivalent biological activity, function, or expression characteristics to the disclosed sequences are encompassed by the present invention, including sequences that exhibit equivalent functional properties, binding properties, folding properties, and/or configurations.

It is further contemplated that specific point mutations disclosed herein in accordance with a sequence according to one embodiment may be combined in various permutations to generate additional sequence variants that retain the functional properties of the disclosed sequences. Accordingly, one or more point mutations described in a given sequence may be combined with one or more point mutations from another disclosed sequence to produce a variant that remains within the scope of the invention. Such combinations may result in sequences having modified but functionally equivalent characteristics, including but not limited to altered stability, expression levels, or functional activity as described herein. Variants generated by combining disclosed mutations while maintaining at least 70%, 75%, 80%, 85%, 90%, 91%, 92%, 93%, 94%, 95%, 96%, 97%, 98%, or 99% sequence identity to an original disclosed sequence are expressly encompassed within the invention.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference). In a specific example, substantially equivalent nucleotide sequences can include sequences that differ by 1, 2, 3, 4, 5, or 6 nucleotides. In a specific example, substantially equivalent amino acid sequences can include sequences that differ by 1, 2, 3, 4, 5, or 6 amino acids.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

1. An inducible promoter system, comprising:
a gene of interest;
an operator site;
a first nucleotide sequence encoding a reverse cumate transcriptional activator; and
a second nucleotide sequence encoding a cumate repressor;
wherein the cumate repressor binds to the operator site to repress transcription of the gene of interest; and
wherein the reverse transcriptional activator binds to the operator site in the presence of an activator to activate transcription of the gene of interest.

2. The inducible promoter system of claim 1, wherein the activator comprises a small molecule.

3. The inducible promoter system of claim 2, wherein the activator comprises cumate.

4. The inducible promoter system of claim 3, wherein the first nucleotide sequence includes a first CymR sequence and the second nucleotide sequence includes a second CymR sequence; and
wherein the first CymR sequence and the second CymR sequence are the same.

5. The inducible promoter system of claim 3, wherein the first nucleotide sequence includes a first CymR sequence and the second nucleotide sequence includes a second CymR sequence; and
wherein the first CymR sequence and the second CymR sequence are different.

6. The inducible promoter system of claim 5, wherein the first CymR sequence and the second CymR sequence are derived from different bacterial species.

7. The inducible promoter system of claim 5, wherein the first CymR sequence and the second CymR sequence are derived from different bacterial strains of the same bacterial species.

8. The inducible promoter system of claim 3, wherein the first nucleotide sequence includes a first CymR sequence encoding a first dimerization domain and a first DNA binding domain;
wherein the second nucleotide sequence includes a second CymR sequence encoding a first dimerization domain and a first DNA binding domain; and
wherein the first DNA binding domain and the second DNA binding domain are the same.

9. The inducible promoter system of claim 8, wherein the first dimerization domain and the second dimerization domain are different.

10. The inducible promoter system of claim 9, wherein the first dimerization domain and the second dimerization domain are derived from different bacterial species.

11. The inducible promoter system of claim 9, wherein the first dimerization domain and the second dimerization domain are derived from different bacterial strains of the same bacterial species.

12. The inducible promoter system of claim 1, wherein the first nucleotide sequence comprises the nucleotide sequence set forth in SEQ ID NO: 1.

13. The inducible promoter system of claim 1, wherein the second nucleotide sequence comprises the nucleotide sequence set forth in one of SEQ ID NO: 2, SEQ ID NO: 3, SEQ ID NO: 4, SEQ ID NO: 5, and SEQ ID NO: 6.

14. The inducible promoter system of claim 1, wherein the first nucleotide sequence comprises the nucleotide sequence set forth in SEQ ID NO: 1; and
wherein the second nucleotide sequence comprises the nucleotide sequence set forth in one of SEQ ID NO: 2, SEQ ID NO: 3, SEQ ID NO: 4, SEQ ID NO: 5, and SEQ ID NO: 6.

15. The inducible promoter system of claim 1, wherein the first nucleotide sequence comprises the nucleotide sequence set forth in SEQ ID NO: 1; and
wherein the second nucleotide sequence comprises the nucleotide sequence set forth in SEQ ID NO: 4.

16. The inducible promoter system of claim 1, comprising a first inducible promoter subsystem and a second inducible promoter subsystem;
wherein the gene of interest comprises a first gene of interest, the operator site comprises a first operator site, and the activator comprises a first activator;
wherein the first inducible promoter subsystem comprises the first gene of interest, the first operator site, the first nucleotide sequence, and the second nucleotide sequence; and
wherein the second inducible promoter subsystem comprises:
a second gene of interest;
a second operator site;
a third nucleotide sequence encoding a transcription activator; and
a second nucleotide sequence encoding a transcription repressor;
wherein the transcription repressor binds to the second operator site to repress transcription of the second gene of interest; and
wherein the transcription activator binds to the second operator site in the presence of a second activator to activate transcription of the second gene of interest.

17. The inducible promoter system of claim 16, wherein the second activator comprises doxycycline.

18. The inducible promoter system of claim 17, wherein the first inducible promoter subsystem and the second inducible promoter subsystem are contained within the same cell.

19. The inducible promoter system of claim 17, wherein the first inducible promoter subsystem and the second inducible promoter subsystem are contained within different cells.

20. The inducible promoter system of claim 16, further comprising a first vector comprising the first inducible promoter subsystem and a second vector comprising the second inducible promoter subsystem.


SEQUENCE LISTING (19 sequences)

SEQ ID NO: 1 - DNA, 744 bp, synthetic construct
  source 1..744: mol_type = other DNA
atggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccggg

SEQ ID NO: 2 - DNA, 618 bp, Pseudomonas putida
  source 1..618: mol_type = other DNA
atggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaagctggagcttctgctcgccacttttgaatggctgtacgaacagatcaccgaaaggagtcgggctcgattagcgaaattgaagccagaggatgacgtaatccagcaaatgctggacgacgccgccgaatttttcctcgatgatgacttctctatatctctagatttgattgtggctgccgaccgggaccctgccttacgcgagggtattcagcgcacggtagagaggaatcggtttgtggtcgaggatatgtggcttggtgttctggtgagccgaggcctctcacgtgatgacgcagaagatatcctttggttgatattcaatagcgtgagagggctggctgtgcgtagcctatggcagaaggacaaagaaaggtttgagcgcgtcaggaacagtaccctcgaaattgcccgagagcggtatgccaaattcaagcgc

SEQ ID NO: 3 - DNA, 831 bp, synthetic construct
  source 1..831: mol_type = other DNA
atggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaagctggagcttctgctcgccacttttgaatggctgtacgaacagatcaccgaaaggagtcgggctcgattagcgaaattgaagccagaggatgacgtaatccagcaaatgctggacgacgccgccgaatttttcctcgatgatgacttctctatatctctagatttgattgtggctgccgaccgggaccctgccttacgcgagggtattcagcgcacggtagagaggaatcggtttgtggtcgaggatatgtggcttggtgttctggtgagccgaggcctctcacgtgatgacgcagaagatatcctttggttgatattcaatagcgtgagagggctggctgtgcgtagcctatggcagaaggacaaagaaaggtttgagcgcgtcaggaacagtaccctcgaaattgcccgagagcggtatgccaaattcaagcgcccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 4 - DNA, 840 bp, synthetic construct
  source 1..840: mol_type = other DNA
atgattaaaaaagtgccagaaaggagacgcacccaggctgaaagacgtgaggaaactaggagtagaatattagacgcagccgtcagcgagctaatgaataaaggatacgccgggttccgggtcgaccaagttgcagccactgccagggtatcccgaggtgcccaaacccatcattttcctaccaaggaatccctcgtactggcggcgcttcagagactttaccaggcttcaaccgaggccagcatgacactcatcgagggattagagtccggcgacgatgtgctggatgccctgatgcgggactcggccagtttctacctgggaccgaattttatcattgctgtgtctatgctcaatcttggcgatcacgaacccgggctgaggcagaaagttagagctatctctcgtaagcaccggttgccagtcgagaaggcttggctgcaggcactgttggactctggcctggccgaagagcctgcaaaaacggtgctcaacataacacaaagcatctatcgcggtatggtgatgcgcaagttcttgaggaacgatcccgaatatacacgatttacaactgagcagtggtcaaagattgcacgagcatatatgaacctaaatgctccttcacggccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 5 - DNA, 837 bp, synthetic construct
  source 1..837: mol_type = other DNA
atggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaaggaatccctcgtactggcggcgcttcagagactttaccaggcttcaaccgaggccagcatgacactcatcgagggattagagtccggcgacgatgtgctggatgccctgatgcgggactcggccagtttctacctgggaccgaattttatcattgctgtgtctatgctcaatcttggcgatcacgaacccgggctgaggcagaaagttagagctatctctcgtaagcaccggttgccagtcgagaaggcttggctgcaggcactgttggactctggcctggccgaagagcctgcaaaaacggtgctcaacataacacaaagcatctatcgcggtatggtgatgcgcaagttcttgaggaacgatcccgaatatacacgatttacaactgagcagtggtcaaagattgcacgagcatatatgaacctaaatgctccttcacggccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 6 - DNA, 834 bp, synthetic construct
  source 1..834: mol_type = other DNA
atgattaaaaaagtgccagaaaggagacgcacccaggctgaaagacgtgaggaaactaggagtagaatattagacgcagccgtcagcgagctaatgaataaaggatacgccgggttccgggtcgaccaagttgcagccactgccagggtatcccgaggtgcccaaacccatcattttcctaccaagctggagcttctgctcgccacttttgaatggctgtacgaacagatcaccgaaaggagtcgggctcgattagcgaaattgaagccagaggatgacgtaatccagcaaatgctggacgacgccgccgaatttttcctcgatgatgacttctctatatctctagatttgattgtggctgccgaccgggaccctgccttacgcgagggtattcagcgcacggtagagaggaatcggtttgtggtcgaggatatgtggcttggtgttctggtgagccgaggcctctcacgtgatgacgcagaagatatcctttggttgatattcaatagcgtgagagggctggctgtgcgtagcctatggcagaaggacaaagaaaggtttgagcgcgtcaggaacagtaccctcgaaattgcccgagagcggtatgccaaattcaagcgcccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 7 - DNA, 326 bp, synthetic construct
  source 1..326: mol_type = other DNA
ggtaccgagcaacaaacagacaatctggtctgtttgtaaacaaacagacaatctggtctgtttgtaaacaaacagacaatctggtctgtttgtaaacaaacagacaatctggtctgtttgtaaacaaacagacaatctggtctgtttgtaaacaaacagacaatctggtctgtttgtaaacaaacagacaatctggtctgtttgtatcgacctatataagcagagctcgtttagtgaaccgtcagatcgcctggagacgccatccacgctgttttgacctccatagaagacaccgggaccgatccagcctccgcggccccgaattg

SEQ ID NO: 8 - DNA, 312 bp, synthetic construct
  source 1..312: mol_type = other DNA
ggtaccgagcagaaacaaaccaacctgtctgtattaagaaacaaaccaacctgtctgtattaagaaacaaaccaacctgtctgtattaagaaacaaaccaacctgtctgtattaagaaacaaaccaacctgtctgtattaagaaacaaaccaacctgtctgtattaagaaacaaaccaacctgtctgtattatcgacctatataagcagagctcgtttagtgaaccgtcagatcgcctggagacgccatccacgctgttttgacctccatagaagacaccgggaccgatccagcctccgcggccccgaattg

SEQ ID NO: 9 - DNA, 326 bp, synthetic construct
  source 1..326: mol_type = other DNA
ggtaccgagcaacaaacagaccgtacagtctgtttgtaaacaaacagaccgtacagtctgtttgtaaacaaacagaccgtacagtctgtttgtaaacaaacagaccgtacagtctgtttgtaaacaaacagaccgtacagtctgtttgtaaacaaacagaccgtacagtctgtttgtaaacaaacagaccgtacagtctgtttgtatcgacctatataagcagagctcgtttagtgaaccgtcagatcgcctggagacgccatccacgctgttttgacctccatagaagacaccgggaccgatccagcctccgcggccccgaattg

SEQ ID NO: 10 - DNA, 312 bp, synthetic construct
  source 1..312: mol_type = other DNA
ggtaccgagcataaacagacgggctagtcggtttgtataaacagacgggctagtcggtttgtataaacagacgggctagtcggtttgtataaacagacgggctagtcggtttgtataaacagacgggctagtcggtttgtataaacagacgggctagtcggtttgtataaacagacgggctagtcggtttgttcgacctatataagcagagctcgtttagtgaaccgtcagatcgcctggagacgccatccacgctgttttgacctccatagaagacaccgggaccgatccagcctccgcggccccgaattg

SEQ ID NO: 11 - DNA, 744 bp, synthetic construct
  source 1..744: mol_type = other DNA
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 12 - DNA, 846 bp, synthetic construct
  source 1..846: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 13 - DNA, 424 bp, synthetic construct
  source 1..424: mol_type = other DNA
caattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacc

SEQ ID NO: 14 - DNA, 10395 bp, synthetic construct
  source 1..10395: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggaagcggagagggcaggggaagtcttctaacatgcggggacgtggaggaaaatcccggccccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 15 - DNA, 9388 bp, synthetic construct
  source 1..9388: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgatacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgttgctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 16 - DNA, 10069 bp, synthetic construct
  source 1..10069: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaagctggagcttctgctcgccacttttgaatggctgtacgaacagatcaccgaaaggagtcgggctcgattagcgaaattgaagccagaggatgacgtaatccagcaaatgctggacgacgccgccgaatttttcctcgatgatgacttctctatatctctagatttgattgtggctgccgaccgggaccctgccttacgcgagggtattcagcgcacggtagagaggaatcggtttgtggtcgaggatatgtggcttggtgttctggtgagccgaggcctctcacgtgatgacgcagaagatatcctttggttgatattcaatagcgtgagagggctggctgtgcgtagcctatggcagaaggacaaagaaaggtttgagcgcgtcaggaacagtaccctcgaaattgcccgagagcggtatgccaaattcaagcgcggaagcggagagggcaggggaagtcttctaacatgcggggacgtggaggaaaatcccggccccatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgatacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgttgctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 17 - DNA, 10282 bp, synthetic construct
  source 1..10282: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaagctggagcttctgctcgccacttttgaatggctgtacgaacagatcaccgaaaggagtcgggctcgattagcgaaattgaagccagaggatgacgtaatccagcaaatgctggacgacgccgccgaatttttcctcgatgatgacttctctatatctctagatttgattgtggctgccgaccgggaccctgccttacgcgagggtattcagcgcacggtagagaggaatcggtttgtggtcgaggatatgtggcttggtgttctggtgagccgaggcctctcacgtgatgacgcagaagatatcctttggttgatattcaatagcgtgagagggctggctgtgcgtagcctatggcagaaggacaaagaaaggtttgagcgcgtcaggaacagtaccctcgaaattgcccgagagcggtatgccaaattcaagcgcccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggaagcggagagggcaggggaagtcttctaacatgcggggacgtggaggaaaatcccggccccatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgatacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgttgctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 18 - DNA, 10291 bp, synthetic construct
  source 1..10291: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgattaaaaaagtgccagaaaggagacgcacccaggctgaaagacgtgaggaaactaggagtagaatattagacgcagccgtcagcgagctaatgaataaaggatacgccgggttccgggtcgaccaagttgcagccactgccagggtatcccgaggtgcccaaacccatcattttcctaccaaggaatccctcgtactggcggcgcttcagagactttaccaggcttcaaccgaggccagcatgacactcatcgagggattagagtccggcgacgatgtgctggatgccctgatgcgggactcggccagtttctacctgggaccgaattttatcattgctgtgtctatgctcaatcttggcgatcacgaacccgggctgaggcagaaagttagagctatctctcgtaagcaccggttgccagtcgagaaggcttggctgcaggcactgttggactctggcctggccgaagagcctgcaaaaacggtgctcaacataacacaaagcatctatcgcggtatggtgatgcgcaagttcttgaggaacgatcccgaatatacacgatttacaactgagcagtggtcaaagattgcacgagcatatatgaacctaaatgctccttcacggccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggaagcggagagggcaggggaagtcttctaacatgcggggacgtggaggaaaatcccggccccatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgatacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgttgctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 19 - DNA, 10288 bp, synthetic construct
  source 1..10288: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttctcggatacccttactctgttgaaaacgaatagataggttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtcattatgtcccctaagagaagaacacaagcagagagggcaatggagacacagggcaaactgattgcagctgccctgggggttctgagagaaaaaggctacgcgggattcagaatcgcagatgtgcccggggctgctggagtctccagaggagcacagtcacaccattttcccactaaggaatccctcgtactggcggcgcttcagagactttaccaggcttcaaccgaggccagcatgacactcatcgagggattagagtccggcgacgatgtgctggatgccctgatgcgggactcggccagtttctacctgggaccgaattttatcattgctgtgtctatgctcaatcttggcgatcacgaacccgggctgaggcagaaagttagagctatctctcgtaagcaccggttgccagtcgagaaggcttggctgcaggcactgttggactctggcctggccgaagagcctgcaaaaacggtgctcaacataacacaaagcatctatcgcggtatggtgatgcgcaagttcttgaggaacgatcccgaatatacacgatttacaactgagcagtggtcaaagattgcacgagcatatatgaacctaaatgctccttcacggccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggggaagcggagagggcaggggaagtcttctaacatgcggggacgtggaggaaaatcccggccccatggtcattatgtcccctaagagaagaactcaggcagagcgtgctatggaaacccagggcaagctgatcgccgctgccctgggcgtgctgagggagaagggctacgctggcttccggatcgccgacgtgcctggagctgctggcgtgagcaggggagctcagtctcaccatttcccaaccaagctggagctgctgctggccacatttgagtggctgtatgagcagatcaccgagagatcccgcgctaggctggctaagctgaagccagaggacgatgtgatccagcagatgctggacgatgctgccgagttctttctggacgatgacttttccatcagcctggacctgatcgtggctgctgaccgcgatcctgtgctgagggagggcatccagcggacagtggagcggaacagattcgtggtgggcgacatctggctgggcgtgctggtgtctcggggcctgtccagagatgacgctgaggatatcctgtggctgatctttaactccgtgagaggcctggccgtgcgcagcctgtggcagaaggataaggagagattcgagagggtgaggaactccaccctggagatcgctcgggagagatacgccaagttcaagcgcgggccggccgacgcccttgacgattttgacttagacatgctcccagccgatgcccttgacgactttgaccttgatatgctgcctgctgacgctcttgacgattttgaccttgacatgctccccgggggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagccacgaacttctctctgttaaagcaagcaggagatgttgaagaaaaccccgggcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaagtttgtacaaaaaagcaggctaggttctgttaagtaactgaacccaatgtcgttagtgacgcttacctcttaagaggtcactgacctaacataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcacccagctttcttgtacaaagtggcaattcggggccgcggaggctggatcggtcccggtgtcttctatggaggtcaaaacagcgtggatggcgtctccaggcgatctgacggttcactaaacgagctctgcttatataggtcgatacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgtttacaaacagaccagattgtctgtttgttgctcggtaccactttatctgagaatagtcaatcttcggaaatcccaggtggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttcaactttattatacatagttgcctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt